(19) United States
(12) Patent Application Publication
(10) Pub. No.: US 2008/0075779 A1
(43) Pub. Date: Mar. 27, 2008

(54) Additives And Methods For Enhancing Active Agent Elution Kinetics

(76) Inventor: Ralph A. Chappa, Prior Lake, MN (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(21) Appl. No.: 11/862,868

(22) Filed: Sep. 27, 2007

Related U.S. Application Data
(60) Provisional application No. 60/827,180, filed on Sep. 27, 2006.

(51) Int. Cl. A61K 9/10; A61K 31/56; A61K 47/30

(52) U.S. Cl. 424/486; 514/182; 514/772.3

(57) ABSTRACT

The present invention relates to methods and additives for increasing active agent elution rates from elution control coatings and elution control coatings including the same. In an embodiment the invention includes an elution control coating comprising a polymeric matrix and at least about 0.1 wt. % of an additive dispersed within the polymeric matrix. The additive can include polyvinylpyrrolidone or a copolymer thereof. An active agent can be dispersed within the polymeric matrix, the active agent having limited solubility in water. Embodiments of the invention also include a method of forming an elution control coating with accelerated release kinetics. Other embodiments are also included herein.

This application claims the benefit of U.S. Provisional Application No. 60/827,180, filed Sep. 27, 2006, the content of which is herein incorporated by reference.

### FIELD OF THE INVENTION

The present invention relates to elution control coatings and additives and methods for enhancing active agent elution rates from elution control coatings.

### BACKGROUND OF THE INVENTION

Active agent elution control coatings are now commonly used in order to deliver active agents to tissues of the body. Elution control coatings can enable the delivery of an active agent over a period of time in order to optimize therapeutic effect. In addition, when disposed on a medical device, elution control coatings can enable site-specific active agent delivery because the medical device can be positioned as desired within the body of a patient.

In some circumstances, it may be desirable to deliver an active agent relatively slowly. In other circumstances, it may be desirable to deliver an active agent relatively quickly. Therefore, a given coating system designed for one application may not provide an elution rate that is desirable for another application. However, once a particular coating system has gone through extensive clinical testing and is well characterized, it can be advantageous to use the same or a similar coating system in other applications, because of a reduced risk of encountering unforeseen adverse effects. For at least this reason, where a given coating exhibits elution kinetics that are too slow for a specific application, it can be desirable to manipulate that coating in order to speed up the elution kinetics.

Accordingly, there is a need for additives and methods for enhancing the elution kinetics of active agents from elution control coatings and elution control coatings incorporating the same.

### SUMMARY OF THE INVENTION

The present invention relates to methods and additives for enhancing active agent elution kinetics of elution control coatings and elution control coatings including the same. In an embodiment the invention includes an elution control coating comprising a polymeric matrix, at least about 0.1 wt. % of an additive dispersed within the polymeric matrix, and an active agent dispersed within the polymeric matrix. The additive can include polyvinylpyrrolidone or a copolymer thereof. The active agent can have limited solubility in water.

In an embodiment the invention includes a method of forming an elution control coating with accelerated release kinetics. The method can include disposing a hydrophobic polymer, an additive comprising polyvinylpyrrolidone, and an active agent having limited solubility in water, on a substrate.

In an embodiment the invention includes a method of increasing the elution rate of an active agent from a coating. The method can include combining a matrix forming polymer, an active agent having limited solubility in water, and a solvent together to form a coating solution. The method can further include adding polyvinylpyrrolidone to the coating solution in an amount sufficient for the coating solution to have a solids concentration of at least about 0.1 wt. % of polyvinylpyrrolidone. The method can further include disposing the coating solution on a substrate.

In an embodiment the invention includes a method of forming an elution control coating. The method can include depositing a polymeric matrix onto a substrate, depositing an active agent onto the substrate, the active agent having limited solubility in water; and depositing polyvinylpyrrolidone onto the substrate in an amount sufficient for the elution control coating to comprise at least about 0.1 wt. % (solids) of polyvinylpyrrolidone.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE FIGURES

The invention may be more completely understood in connection with the following drawings, in which:

FIG. 1 is a graph showing elution of estradiol from a coating into a test solution over time.

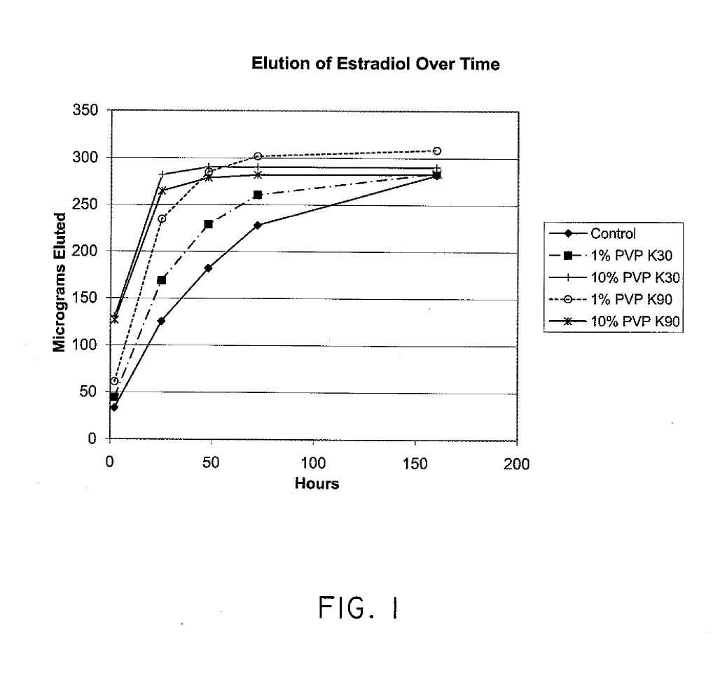

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The embodiments of the present invention described herein are not intended to be exhaustive or to limit the invention to the precise forms disclosed in the following detailed description. Rather, the embodiments are chosen and described so that those skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Sometimes it can be desirable to increase or accelerate the elution kinetics of an active agent from a particular elution control coating in order to deliver the active agent at a more optimal rate. Methods of embodiments herein can include increasing the elution rate of an active agent from an elution control coating by using an additive that functions to increase or accelerate the elution kinetics of active agents. An exemplary additive used with various embodiments herein is polyvinylpyrrolidone (PVP). However, embodiments can also include the use of other additives.

Some active agents have limited solubility in water or are insoluble in water. These active agents can present challenges in the contexts of both depositing them onto substrates and then eluting them from coatings after deposition. However, the presence of PVP in a matrix can effectively increase or accelerate the elution rate of water insoluble active agents from polymeric matrices. In an embodiment, the invention includes an elution control coating including a polymeric matrix and polyvinylpyrrolidone (PVP).

Some active agents are relatively insoluble in both polar and non-polar solvents. It can be particularly challenging to achieve desirable elution kinetics with these types of active agents. However, additives described herein, such as PVP, can be used to achieve desirable elution profiles with active agents that are relatively insoluble in both polar and non-polar solvents.

Some types of active agents assume a crystalline form more readily than others. For example, estradiol, a steroid, readily crystallizes under supersaturated conditions. Crystalline forms are generally thermodynamically stable and therefore generally go into solution less readily than otherwise identical compounds in amorphous form. Some elution processes depend on the active agent first becoming solubilized in a solvent. As such, while not intending to be bound by theory, it is believed that some additives, such as PVP, can accelerate the elution kinetics of some active agents by inhibiting crystallization. However, it is believed that additives can also impact the elution kinetics of some active agents through other mechanisms.

Cross-links are covalent bonds linking one polymer chain to another. Cross-linking of polymers generally changes physical characteristics such as making polymers more rigid and resistant to physical deformation. However, cross-linking of polymers can also make them susceptible to cracking and fracturing. Cracking and fracturing of an elution control coating can be undesirable as it can lead to pieces of the coating separating from the medical device. In addition, cross-linking of a matrix can inhibit passage of molecules through the matrix making it more difficult or impossible to pass active agent molecules through the matrix. In an embodiment, the invention includes an elution control coating comprising an uncross-linked matrix.

In some embodiments, elution control coatings of the invention are disposed on medical devices that are designed to be explanted after a period of residence within the body of a patient. The process of inserting and removing a medical device from a patient may result in various stresses being applied to the elution control coating. Generally, it is undesirable for portions of the elution control coating to separate from the medical device and remain within the patient after explant of the medical device. As such, embodiments of the invention can include elution control coatings that are configured to retain their structural integrity for a period of time within the body. In an embodiment, the elution control coating is configured to retain structural integrity for at least about six weeks. In an embodiment, the elution control coating is configured to resist fracturing under in vivo conditions for at least about six weeks.

In some embodiments of the invention, elution control coatings can include a polymeric matrix including one or more polymers and an additive. In some cases, the elution control coating can include a polymeric matrix, an additive, and an active agent. The polymeric matrix can provide structural integrity to the elution control coating in addition to retaining the active agent prior to elution.

Components of the elution control coating can be applied onto a substrate. In some cases, the components of the coating are mixed with a solvent and then applied onto a substrate. In an embodiment, a polymer (or polymers) that will form the matrix (“matrix polymer”) of the finished coating, an active agent, and an additive are mixed with a solvent to form a coating solution or mixture that is then applied to a substrate to form an elution control coating. However, it will be appreciated that some or all of the components may be applied as a separate solutions or mixtures. By way of example, the matrix polymer(s) can be mixed with a first solvent to form a first solution while the active agent and the additive can be mixed with a second solvent to form a second solution. Then both solutions can be applied separately to a substrate to form the elution control coating. In some embodiments, the additive is mixed into the same solution as the matrix polymer. In other embodiments, the additive is mixed into the same solution as the active agent. In still other embodiments, the additive is mixed with a solvent to form a separate solution that is applied to a substrate independently of the active agent and the matrix polymer.

The one or more polymers (matrix polymers) forming the polymeric matrix of the elution control coating can be degradable or non-degradable. In some embodiments, the matrix polymer can include a combination of degradable and non-degradable polymers. Degradable and non-degradable polymers are described in further detail below. In embodiments where the matrix polymer includes at least two different polymers, such as a degradable and non-degradable polymer, the two polymers can be applied together from one solution containing both or they can be applied as two different solutions. In some cases, the matrix polymer includes two different polymers with different solubility properties. In such an embodiment, a first polymer in a relatively polar solvent can be applied separately from a second polymer in a relatively non-polar solvent. In embodiments where separate solutions are applied to a substrate to form the elution control coating they can be applied simultaneously, substantially simultaneously, sequentially, or in any other manner desired.

The solutions that form the elution control coating (coating solutions) can be applied onto a substrate using any of a variety of coating techniques including dip-coating, spray-coating (including both gas-atomization and ultrasonic atomization), fogging, brush coating, press coating, blade coating, and the like. The coating solutions can be applied under conditions where atmospheric characteristics such as relative humidity, temperature, gaseous composition, and the like are controlled.

In some embodiments, the coating solutions are applied using a spray technique. The coating solutions can be applied using a spraying apparatus including an ultrasonic spray nozzle. Exemplary spray coating equipment that can be used to apply components of the invention can be found in U.S. Pat. No. 6,562,136; U.S. Pat. No. 7,077,910; U.S. Pub. App. No. US 2004/0062875; U.S. Pub. App. No. 2005/0158449; U.S. Pub. App. No. 2006/0088653; U.S. Pub. App. No. 2005/0196424; and U.S. Pub. App. No. 2007/0128343, the contents of which are all hereby incorporated by reference.

### Additives

Additives of the invention can include compounds that function to increase or accelerate the elution kinetics of an elution control coating in comparison to an otherwise identical coating lacking the additive. Additives used in embodiments of the invention can also include those compounds that are biocompatible and substantially non-thrombogenic. Additives of the invention can specifically include polyvinylpyrrolidone (PVP) (CAS No. 9003-39-8) (homopolymer of N-vinyl-2-pyrrolidone). In some embodiments, the polyvinylpyrrolidone used can have a molecular weight of between about 5,000 Daltons and about 400,000 Daltons. Additives of the invention can also include co-polymers of polyvinylpyrrolidone. In some embodiments, additives can also include one or more of polyvinylcaprolactam, polyethylene glycol (PEG), polyethylene oxide, polyvinyl alcohol, and polyacrylamide.

Additives of the invention can include polymers having both hydrophilic and hydrophobic properties (amphiphilic polymers). Such amphiphilic polymers can have a solubility enhancing effect on poorly soluble active agents similar to the effects of a surfactant. While not intending to be bound by theory, it is believed that the surfactant-like effects of amphiphilic polymers can function to increase the elution rate of active agents that are relatively insoluble in both polar and non-polar solvents. It is further believed that the use of polymers, such as PVP and PEG, as an amphiphilic additive to increase the elution rate of an active agent from a coating can offer advantages in comparison to the use of traditional surfactants such as sodium dodecyl sulphate. Specifically, it has been observed some traditional surfactants have a deleterious effect on the appearance and/or structure of elution control coatings.

Additives can be present in the elution control coating in an amount sufficient to increase the elution kinetics of the elution control coating. In an embodiment, the elution control coating includes at least about 0.1 wt. % additive (of total solids). In an embodiment, the elution control coating includes at least about 5.0 wt. % additive (of total solids). In an embodiment, the elution control coating includes at least about 10.0 wt. % additive (of total solids).

The additive or additives can be dispersed within the polymeric matrix of the elution control coating. As used herein, the term “dispersed” shall refer to the property of being distributed in a soluble or insoluble state. In some embodiments, the additive is part of the same phase as the matrix polymers. In other embodiments, the additive is part of a phase separate from the matrix polymers.

### Non-Degradable Matrix Polymers

In some embodiments of the invention, the polymeric matrix can include one or more non-degradable (durable) polymers. In an embodiment, the non-degradable polymer includes a plurality of polymers, including a first polymer and a second polymer. When the coating solution contains only one polymer, it can be either a first or second polymer as described herein. As used herein, term “(meth)acrylate” when used in describing polymers shall mean the form including the methyl group (methacrylate) or the form without the methyl group (acrylate).

First polymers of the invention can include a polymer selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively). An exemplary first polymer is poly(n-butyl methacrylate) (pBMA). Such polymers are available commercially, e.g., from Aldrich, with molecular weights ranging from about 200,000 Daltons to about 320,000 Daltons, and with varying inherent viscosity, solubility, and form (e.g., as crystals or powder). In some embodiments, poly(n-butyl methacrylate) (pBMA) is used with a molecular weight of about 200,000 Daltons to about 300,000 Daltons.

Examples of suitable first polymers also include polymers selected from the group consisting of poly(aryl(meth)acrylates), poly(aralkyl (meth)acrylates), and poly(aryloxyalkyl(meth)acrylates). Such terms are used to describe polymeric structures wherein at least one carbon chain and at least one aromatic ring are combined with acrylic groups, typically esters, to provide a composition. In particular, exemplary polymeric structures include those with aryl groups having from 6 to 16 carbon atoms and with weight average molecular weights from about 50 to about 900 kilodaltons. Suitable poly(aralkyl(meth)acrylates), poly(arylalky(meth)acrylates) or poly(aryloxyalkyl (meth)acrylates) can be made from aromatic esters derived from alcohols also containing aromatic moieties. Examples of poly(aryl(meth)acrylates) include poly(9-anthracenyl methacrylate), poly(chlorophenylacrylate), poly(methacryloxy-2-hydroxybenzophenone), poly(methacryloxybenzotriazole), poly(naphthylacrylate) and -methacrylate), poly(4-nitrophenyl acrylate), poly(pentachloro(bromo, fluoro) acrylate) and -methacrylate), and poly(phenyl acrylate) and -methacrylate). Examples of poly(aralkyl (meth)acrylates) include poly(benzyl acrylate) and -methacrylate), poly(2-phenethyl acrylate) and -methacrylate, and poly(1-pyrenylmethyl methacrylate). Examples of poly(aryloxyalkyl (meth)acrylates) include poly(phenoxyethyl acrylate) and -methacrylate), and poly(polyethylene glycol phenyl ether acrylates) and -methacrylates with varying polyethylene glycol molecular weights.

Examples of suitable second polymers are available commercially and include poly(ethylene-co-vinyl acetate) (pEVA) having vinyl acetate concentrations of between about 10% and about 50% (12%, 14%, 18%, 25%, 33% versions are commercially available), in the form of beads, pellets, granules, etc. pEVA co-polymers with lower percent vinyl acetate become increasingly insoluble in typical solvents, whereas those with higher percent vinyl acetate become decreasingly durable.

An exemplary polymer mixture includes mixtures of pBMA and pEVA. This mixture of polymers can be used with absolute polymer concentrations (i.e., the total combined concentrations of both polymers in the coating material), of between about 0.25 wt. % and about 99 wt. %. This mixture can also be used with individual polymer concentrations in the coating solution of between about 0.05 wt. % and about 99 wt. %. In one embodiment the polymer mixture includes pBMA with a molecular weight of from 100 kilodaltons to 900 kilodaltons and a pEVA copolymer with a vinyl acetate content of from 1 to 99 weight percent. In an embodiment the polymer mixture includes pBMA with a molecular weight of from 200 kilodaltons to 300 kilodaltons and a pEVA copolymer with a vinyl acetate content of from 24 to 36 weight percent. The concentration of the active agent or agents dissolved or suspended in the coating mixture can range from 0.01 to 99 percent, by weight, based on the weight of the final coating material.

Second polymers can also comprise one or more polymers selected from the group consisting of (i) poly(alkylene-co-alkyl(meth)acrylates, (ii) ethylene copolymers with other alkylenes, (iii) polybutenes, (iv) diolefin derived non-aromatic polymers and copolymers, (v) aromatic group-containing copolymers, and (vi) epichlorohydrin-containing polymers.

Poly(alkylene-co-alkyl(meth)acrylates) include those copolymers in which the alkyl groups are either linear or branched, and substituted or unsubstituted with non-interfering groups or atoms. Such alkyl groups can comprise from 1 to 8 carbon atoms, inclusive. Such alkyl groups can comprise from 1 to 4 carbon atoms, inclusive. In an embodiment, the alkyl group is methyl. In some embodiments, copolymers that include such alkyl groups can comprise from about 15% to about 80% (wt) of alkyl acrylate. When the alkyl group is methyl, the polymer contains from about 20% to about 40% methyl acrylate in some embodiments, and from about 25% to about 30% methyl acrylate in a particular embodiment. When the alkyl group is ethyl, the polymer contains from about 15% to about 40% ethyl acrylate in an embodiment, and when the alkyl group is butyl, the polymer contains from about 20% to about 40% butyl acrylate in an embodiment.

Alternatively, second polymers can comprise ethylene copolymers with other alkylenes, which in turn, can include straight and branched alkylenes, as well as substituted or unsubstituted alkylenes. Examples include copolymers prepared from alkylenes that comprise from 3 to 8 branched or linear carbon atoms, inclusive. In an embodiment, copolymers prepared from alkylene groups that comprise from 3 to 4 branched or linear carbon atoms, inclusive. In a particular embodiment, copolymers prepared from alkylene groups containing 3 carbon atoms (e.g., propene). By way of example, the other alkylene is a straight chain alkylene (e.g., 1-alkylene). Exemplary copolymers of this type can comprise from about 20% to about 90% (based on moles) of ethylene. In an embodiment, copolymers of this type comprise from about 35% to about 80% (mole) of ethylene. Such copolymers will have a molecular weight of between about 30 kilodaltons to about 500 kilodaltons. Exemplary copolymers are selected from the group consisting of poly(ethylene-co-propylene), poly(ethylene-co-1-butene), polyethylene-co-1-butene-co-1-hexene) and/or poly(ethylene-co-1-octene).

“Polybutenes” include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In an embodiment, the polybutene contains at least about 90% (wt) of isobutylene or 1-butene. In a particular embodiment, the polybutene contains at least about 90% (wt) of isobutylene. The polybutene may contain non-interfering amounts of other ingredients or additives, for instance it can contain up to 1000 ppm of an antioxidant (e.g., 2,6-di-tert-butyl-methylphenol). By way of example, the polybutene can have a molecular weight between about 150 kilodaltons and about 1,000 kilodaltons. In an embodiment, the polybutene can have between about 200 kilodaltons and about 600 kilodaltons. In a particular embodiment, the polybutene can have between about 350 kilodaltons and about 500 kilodaltons. Polybutenes having a molecular weight greater than about 600 kilodaltons, including greater than 1,000 kilodaltons are available but are expected to be more difficult to work with.

Additional alternative second polymers include diolefin-derived, non-aromatic polymers and copolymers, including those in which the diolefin monomer used to prepare the polymer or copolymer is selected from butadiene (CH₂═CH—CH═CH₂) and/or isoprene (CH₂═CH—C(CH₃)═CH₂). In an embodiment, the polymer is a homopolymer derived from diolefin monomers or is a copolymer of diolefin monomer with non-aromatic mono-olefin monomer, and optionally, the homopolymer or copolymer can be partially hydrogenated. Such polymers can be selected from the group consisting of polybutadienes prepared by the polymerization of cis-, trans- and/or 1,2-monomer units, or from a mixture of all three monomers, and polyisoprenes prepared by the polymerization of cis-1,4- and/or trans-1,4-monomer units. Alternatively, the polymer is a copolymer, including graft copolymers, and random copolymers based on a non-aromatic mono-olefin monomer such as acrylonitrile, and an alkyl (meth)acrylate and/or isobutylene. In an embodiment, when the mono-olefin monomer is acrylonitrile, the interpolymerized acrylonitrile is present at up to about 50% by weight; and when the mono-olefin monomer is isobutylene, the diolefin is isoprene (e.g., to form what is commercially known as a “butyl rubber”). Exemplary polymers and copolymers have a molecular weight between about 150 kilodaltons and about 1,000 kilodaltons. In an embodiment, polymers and copolymers have a molecular weight between about 200 kilodaltons and about 600 kilodaltons.

Additional alternative second polymers include aromatic group-containing copolymers, including random copolymers, block copolymers and graft copolymers. In an embodiment, the aromatic group is incorporated into the copolymer via the polymerization of styrene. In a particular embodiment, the random copolymer is a copolymer derived from copolymerization of styrene monomer and one or more monomers selected from butadiene, isoprene, acrylonitrile, a C₁-C₄ alkyl (meth)acrylate (e.g., methyl methacrylate) and/or butene. Useful block copolymers include copolymer containing (a) blocks of polystyrene, (b) blocks of an polyolefin selected from polybutadiene, polyisoprene and/or polybutene (e.g., isobutylene), and (c) optionally a third monomer (e.g., ethylene) copolymerized in the polyolefin block. The aromatic group-containing copolymers contain about 10% to about 50% (wt.) of polymerized aromatic monomer and the molecular weight of the copolymer is from about 300 kilodaltons to about 500 kilodaltons. In an embodiment, the molecular weight of the copolymer is from about 100 kilodaltons to about 300 kilodaltons.

Additional alternative second polymers include epichlorohydrin homopolymers and poly(epichlorohydrin-co-alkylene oxide) copolymers. In an embodiment, in the case of the copolymer, the copolymerized alkylene oxide is ethylene oxide. By way of example, epichlorohydrin content of the epichlorohydrin-containing polymer is from about 30% to 100% (wt). In an embodiment, epichlorohydrin content is from about 50% to 100% (wt). In an embodiment, the epichlorohydrin-containing polymers have a molecular weight from about 100 kilodaltons to about 300 kilodaltons.

Non-degradable polymers can also include those described in U.S. Pub. Pat. App. No. 2007/0026037, entitled “DEVICES, ARTICLES, COATINGS, AND METHODS FOR CONTROLLED ACTIVE AGENT RELEASE OR HEMOCOMPATIBILITY”, the contents of which is herein incorporated by reference. As a specific example, non-degradable polymers can include random copolymers of butyl methacrylate-co-acrylamido-methyl-propane sulfonate (BMA-AMPS). In some embodiments, the random copolymer can include acrylamido-methyl-propane sulfonate (AMPS) in an amount equal to about 0.5 mol. % to about 40 mol. %.

### Degradable Matrix Polymers

In some embodiments of the invention, the polymeric matrix can include one or more degradable polymers. Degradable polymers used with embodiments of the invention can include natural or synthetic polymers. The term “degradable” as used herein with reference to polymers, shall refer to those natural or synthetic polymers that break down under physiological conditions into constituent components over a period of time. By way of example, many degradable polymers include hydrolytically unstable linkages in the polymeric backbone. The cleavage of these unstable linkages leads to degradation of the polymer. The terms “erodible”, “bioerodible”, “biodegradable” and “non-durable” shall be used herein interchangeably with the term “degradable”. Degradable polymers can include both natural and synthetic polymers. Examples of degradable polymers can include those with hydrolytically unstable linkages in the polymeric backbone. Degradable polymers of the invention can include both those with bulk erosion characteristics and those with surface erosion characteristics.

Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(B-malonic acid), poly(propylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates); degradable polyiminocarbonates; degradable polyarylates (such as tyrosine-based polyarylates); degradable polyorthoesters; degradable polyurethanes; degradable polyphosphazenes; and degradable polyhydroxyalkanoates; and copolymers thereof.

Natural or naturally-based degradable polymers can include polysaccharides and modified polysaccharides such as starch, cellulose, chitin, chitosan, and copolymers thereof.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)-]x[-O—(CH₂)₄—O—C(O)—C₆H₄—C(O)-]y,

where —C₆H₄— designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. n can be selected such that the molecular weight of the PEG block is between about 300 and about 4000. X and y can be selected so that the multiblock copolymer contains from about 55% up to about 80% PEG by weight. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure.

Degradable polyesteramides can include those formed from the monomers OH-x-OH, z, and COOH-y-COOH, wherein x is alkyl, y is alkyl, and z is leucine or phenylalanine.

Degradable polymeric materials can also be selected from: (a) non-peptide polyamino polymers; (b) polyiminocarbonates; (c) amino acid-derived polycarbonates and polyarylates; and (d) poly(alkylene oxide) polymers.

In an embodiment, the degradable polymeric material is composed of a non-peptide polyamino acid polymer. Exemplary non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, including two or three amino acid units having one of the following two structures illustrated below:

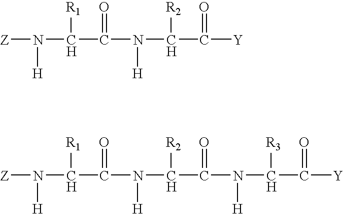

wherein the monomer units are joined via hydrolytically labile bonds at not less than one of the side groups R₁, R₂, and R₃, and where R₁, R₂, R₃ are the side chains of naturally occurring amino acids; Z is any desirable amine protecting group or hydrogen; and Y is any desirable carboxyl protecting group or hydroxyl. Each monomer unit comprises naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are degradable, and can provide zero-order release kinetics for the delivery of active agents in a variety of therapeutic applications. According to these aspects, the amino acids are selected from naturally occurring L-alpha amino acids, including alanine, valine, leucine, isoleucine, proline, serine, threonine, aspartic acid, glutamic acid, asparagine, glutamine, lysine, hydroxylysine, arginine, hydroxyproline, methionine, cysteine, cystine, phenylalanine, tyrosine, tryptophan, histidine, citrulline, ornithine, lanthionine, hypoglycin A, β-alanine, γ-amino butyric acid, a aminoadipic acid, canavanine, venkolic acid, thiolhistidine, ergothionine, dihydroxyphenylalanine, and other amino acids well recognized and characterized in protein chemistry.

Degradable polymers of the invention can also include polymerized polysaccharides such as those described in U.S. Publ. Pat. Application No. 2005/0255142, entitled “COATINGS FOR MEDICAL ARTICLES INCLUDING NATURAL BIODEGRADABLE POLYSACCHARIDES”, U.S. Publ. Pat. Application No. 2007/0065481, entitled “COATINGS INCLUDING NATURAL BIODEGRADABLE POLYSACCHARIDES AND USES THEREOF”, and in U.S. Application No. 60/782,957, entitled “HYDROPHOBIC DERIVATIVES OF NATURAL BIODEGRADABLE POLYSACCHARIDES”, all of which are herein incorporated by reference.

Degradable polymers of the invention can also include dextran based polymers such as those described in U.S. Pat. No. 6,303,148, entitled “PROCESS FOR THE PREPARATION OF A CONTROLLED RELEASE SYTEM”. Exemplary dextran based degradable polymers including those available commercially under the trade name OCTODEX.

Degradable polymers of the invention can further include collagen/hyaluronic acid polymers.

Degradable polymers of the invention can include multi-block copolymers, comprising at least two hydrolysable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolysable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione). The composition of the pre-polymers can be chosen in such a way that the maximum glass transition temperature of the resulting copolymer is below 37° C. at body conditions. To fulfill the requirement of a Tg below 37° C., some of the above-mentioned monomers or combinations of monomers can be more preferred than others. This may by itself lower the Tg, or the pre-polymer is initiated with a polyethylene glycol with sufficient molecular weight to lower the glass transition temperature of the copolymer. The degradable multi-block copolymers can include hydrolysable sequences being amorphous and the segments can be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained.

### Hydrophobic Polymers

In some embodiments of the invention, the polymeric matrix can include one or more hydrophobic polymers. Hydrophobic polymers can include degradable and/or non-degradable polymers. One method of defining the hydrophobicity of a polymer is by the solubility parameter (or Hildebrand parameter) of the polymer. The solubility parameter describes the attractive strength between molecules of the material. The solubility parameter is represented by Equation 1:

δ=(ΔE^{v}/V)^{1/2} (Equation 1)

where δ=solubility parameter ((cal/cm³)^{1/2})

ΔE^{v}=energy of vaporization (cal)

V=molar volume (cm³)

Solubility parameters cannot be calculated for polymers from heat of vaporization data because of their nonvolatility. Accordingly, solubility parameters must be calculated indirectly. One method involves identifying solvents in which a polymer dissolves without a change in heat or volume and then defining the solubility parameter of the polymer to be the same as the solubility parameters of the identified solvents. A more complete discussion of solubility parameters and methods of calculating the same can be found in Brandup et al., Polymer Handbook, 4th Ed., John Wiley & Sons, N.Y. (1999) beginning at VII p. 675.

As a general rule, the value of the solubility parameter 6 is inversely proportional to the degree of hydrophobicity of a polymer. Thus, polymers that are very hydrophobic may have a low solubility parameter value. This general proposition is particularly applicable for polymers having a glass transition temperature below physiological temperature. In an embodiment, hydrophobic polymers used with the invention have a solubility parameter less than about 11.0 (cal/cm³)^{1/2}. In an embodiment, hydrophobic polymers used with the invention have a solubility parameter of less than about 10.0 (cal/cm³)^{1/2}.

### Active Agents

Elution control coatings as described herein can include one or more active agents. As used herein, the term “active agent” means a compound that has a particular desired activity. For example, an active agent can be a therapeutic compound that exerts a specific activity on a subject. In some embodiments, active agent will, in turn, refer to a peptide, protein, carbohydrate, nucleic acid, lipid, polysaccharide or combinations thereof, or synthetic inorganic or organic molecule, that causes a desired biological effect when administered in vivo to an animal, including but not limited to birds and mammals, including humans. In some embodiments, the active agent can be a bioactive agent.

Active agents used with embodiments of the invention can have various solubility properties. By way of example, some active agents can be relatively soluble in polar solvents whereas other active agents can be relatively soluble in non-polar solvents. Active agents used herein can specifically include those that are insoluble in water. As used herein, the phrases “insoluble in water” and “water insoluble” shall refer to a solubility in water of less than about 1.0 grams/liter at 25 degrees Celsius. Active agents used herein can also include those that have limited solubility in water. As used herein, the phrase “limited solubility” shall refer to a solubility in water of less than about 50.0 grams/liter at 25 degrees Celsius. As such the class of limited solubility active agents includes water insoluble active agents.

Active agents used herein can specifically include agents that are relatively insoluble in both polar and non-polar solvents. By way of example, active agents used herein can include those agents having a solubility in water of less than about 2 milligrams per milliliter and a solubility in chloroform of less than about 2 milligrams per milliliter. Active agents used herein can also include those agents having a solubility in water of less than about 1 milligrams per milliliter and a solubility in chloroform of less than about 1 milligrams per milliliter.

Active agents useful according to the invention include substances that possess desirable therapeutic characteristics for application to the implantation site. Active agents useful in the present invention can include many types of therapeutics including thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, antimicrobial agents, antibiotics, antibacterial agents, antiparasite and/or antiprotozoal solutes, antiseptics, antifungals, angiogenic agents, anti-angiogenic agents, inhibitors of surface glycoprotein receptors, antimitotics, microtubule inhibitors, anti-secretory agents, actin inhibitors, remodeling inhibitors, antisense nucleotides, anti-metabolites, miotic agents, anti-proliferatives, anticancer chemotherapeutic agents, anti-neoplastic agents, anti-polymerases, anti-virals, anti-AIDS substances, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, extracellular matrix components, ACE inhibitors, free radical scavengers, chelators, anti-oxidants, photodynamic therapy agents, gene therapy agents, anesthetics, immunotoxins, neurotoxins, opioids, dopamine agonists, hypnotics, antihistamines, tranquilizers, anticonvulsants, muscle relaxants and anti-Parkinson substances, antispasmodics and muscle contractants, anticholinergics, ophthalmic agents, anti-glaucoma solutes, prostaglandins, antidepressants, antipsychotic substances, neurotransmitters, anti-emetics, imaging agents, specific targeting agents, and cell response modifiers.

More specifically, in embodiments the active agent can include heparin, covalent heparin, synthetic heparin salts, or another thrombin inhibitor; hirudin, hirulog, argatroban, D-phenylalanyl-L-poly-L-arginyl chloromethyl ketone, or another antithrombogenic agent; urokinase, streptokinase, a tissue plasminogen activator, or another thrombolytic agent; a fibrinolytic agent; a vasospasm inhibitor; a calcium channel blocker, a nitrate, nitric oxide, a nitric oxide promoter, nitric oxide donors, dipyridamole, or another vasodilator; HYTRIN® or other antihypertensive agents; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; dimethyl sulfoxide (DMSO), a retinoid, or another antisecretory agent; cytochalasin or another actin inhibitor; cell cycle inhibitors; remodeling inhibitors; deoxyribonucleic acid, an antisense nucleotide, or another agent for molecular genetic intervention; an aptamer (such as MACUGEN®); methotrexate, or another antimetabolite or antiproliferative agent; tamoxifen citrate, TAXOL®, paclitaxel, or the derivatives thereof, rapamycin (or other rapalogs e.g. ABT-578 or sirolimus), vinblastine, vincristine, vinorelbine, etoposide, tenopiside, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, chlorambucil, ethylenimines, methylmelamines, alkyl sulfonates (e.g., busulfan), nitrosoureas (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, morpholino phosphorodiamidate oligomer or other anti-cancer chemotherapeutic agents; cyclosporin, tacrolimus (FK-506), pimecrolimus, azathioprine, mycophenolate mofetil, mTOR inhibitors, or another immunosuppressive agent; cortisol, cortisone, dexamethasone, dexamethasone sodium phosphate, dexamethasone acetate, dexamethasone derivatives, betamethasone, fludrocortisone, prednisone, prednisolone, 6U-methylprednisolone, triamcinolone (e.g., triamcinolone acetonide), or another steroidal agent; rosiglitazone; trapidil (a PDGF antagonist), angiopeptin (a growth hormone antagonist), angiogenin, a growth factor (such as vascular endothelial growth factor (VEGF)), or an anti-growth factor antibody (e.g., ranibizumab, which is sold under the tradename LUCENTIS®), or another growth factor antagonist or agonist; dopamine, bromocriptine mesylate, pergolide mesylate, or another dopamine agonist; ⁶⁰Co (5.3 year half life), ¹⁹²Ir (73.8 days), ³²P (14.3 days), ¹¹¹In (68 hours), ⁹⁰Y (64 hours), ⁹⁹Tc (6 hours), or another radiotherapeutic agent; iodine-containing compounds, barium-containing compounds, gold, tantalum, platinum, tungsten or another heavy metal functioning as a radiopaque agent; a peptide, a protein, an extracellular matrix component, a cellular component or another biologic agent; captopril, enalapril or another angiotensin converting enzyme (ACE) inhibitor; angiotensin receptor blockers; enzyme inhibitors (including growth factor signal transduction kinase inhibitors); ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹I-, ³²P- or ³⁶S-radiolabelled form or other radiolabelled form of any of the foregoing; an estrogen (such as estradiol, estriol, estrone, and the like) or another sex hormone; AZT or other antipolymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluorozinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin, or other antibody targeted therapy agents; gene therapy agents; enalapril and other prodrugs; PROSCAR®, HYTRIN® or other agents for treating benign prostatic hyperplasia (BHP); VIAGRA®, mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these.

Other biologically useful compounds that can also be included in the coating include, but are not limited to, hormones, β-blockers, anti-anginal agents, cardiac inotropic agents, corticosteroids, analgesics, anti-inflammatory agents, anti-arrhythmic agents, immunosuppressants, anti-bacterial agents, anti-hypertensive agents, anti-malarials, anti-neoplastic agents, anti-protozoal agents, anti-thyroid agents, sedatives, hypnotics and neuroleptics, diuretics, anti-parkinsonian agents, gastro-intestinal agents, anti-viral agents, anti-diabetics, anti-epileptics, anti-fungal agents, histamine H-receptor antagonists, lipid regulating agents, muscle relaxants, nutritional agents such as vitamins and minerals, stimulants, nucleic acids, polypeptides, and vaccines.

Antibiotics are substances which inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, erythromycin, geldanamycin, geldanamycin analogs, ciprofloxacin, cephalosporins, or the like. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Antiseptics are recognized as substances that prevent or arrest the growth or action of microorganisms, generally in a nonspecific fashion, e.g., either by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Antiviral agents are substances capable of destroying or suppressing the replication of viruses. Examples of anti-viral agents include α-methyl-1-adamantanemethylamine, hydroxy-ethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatechol, kinase inhibitors (such as diacylglycerol kinase inhibitor I and diacylglycerol kinase inhibitor II), 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Local anesthetics are substances that have an anesthetic effect in a localized region. Examples of such anesthetics include procaine, lidocaine, tetracaine and dibucaine.

Imaging agents are agents capable of imaging a desired site, e.g., tumor, in vivo. Examples of imaging agents include substances having a label that is detectable in vivo, e.g., antibodies attached to fluorescent labels. The term antibody includes whole antibodies or fragments thereof.

Cell response modifiers are chemotactic factors such as platelet-derived growth factor (PDGF). Other chemotactic factors include neutrophil-activating protein, monocyte chemoattractant protein, macrophage-inflammatory protein, SIS (small inducible secreted), platelet factor, platelet basic protein, melanoma growth stimulating activity factor, epidermal growth factor, transforming growth factor alpha, fibroblast growth factor, platelet-derived endothelial cell growth factor, insulin-like growth factor, nerve growth factor, bone growth/cartilage-inducing factor (alpha and beta), and matrix metalloproteinase inhibitors. Other cell response modifiers are the interleukins, interleukin receptors, interleukin inhibitors, interferons, including alpha, beta, and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins, antisense molecules, androgenic receptor blockers and statin agents.

In an embodiment, the active agent used with the invention includes compounds having a steroid ring system. Compounds having a steroid ring system can be referred to as steroids. In an embodiment, the active agent is a steroid. Steroids include both naturally occurring compounds and synthetic analogues based on the cyclopenta[a]phenanthrene carbon skeleton, partially or completely hydrogenated. Steroids can include glucocorticoids, estrogens and androgens. By way of example, steroids can include dexamethasone, dexamethasone acetate, dexamethasone sodium phosphate, cortisone, cortisone acetate, hydrocortisone, hydrocortisone acetate, hydrocortisone cypionate, hydrocortisone sodium phosphate, hydrocortisone sodium succinate, prednisone, prednisolone, prednisolone acetate, prednisolone sodium phosphate, prednisolone tebutate, prednisolone pivalate, triamcinolone, triamcinolone acetonide, triamcinolone hexacetonide, triamcinolone diacetate, methylprednisolone, methylprednisolone acetate, methylprednisolone sodium succinate, flunsolide, beclomethasone dipropionate, betamethasone sodium phosphate, betamethasone, vetamethasone disodium phosphate, vetamethasone sodium phosphate, betamethasone acetate, betamethasone disodium phosphate, chloroprednisone acetate, corticosterone, desoxycorticosterone, desoxycorticosterone acetate, desoxycorticosterone pivalate, desoximethasone, estradiol, fluorocortisone, fluorocortisone acetate, dichlorisone acetate, fluorohydrocortisone, fluorometholone, fluprednisolone, paramethasone, paramethasone acetate, androsterone, fluoxymesterone, aldosterone, methandrostenolone, methylandrostenediol, methyl testosterone, norethandrolone, testosterone, testosterone enanthate, testosterone propionate, equilenin, equilin, estradiol benzoate, estradiol dipropionate, estriol, estrone, estrone benzoate, acetoxypregnenolone, anagestone acetate, chlormadinone acetate, fluorogestone acetate, hydroxymethylprogesterone, hydroxymethylprogesterone acetate, hydroxyprogesterone, hydroxyprogesterone acetate, hydroxyprogesterone caproate, melengestrol acetate, normethisterone, pregnenolone, progesterone, ethynyl estradiol, mestranol, dimethisterone, ethisterone, ethynodiol diacetate, norethindrone, norethindrone acetate, norethisterone, fluocinolone acetonide, flurandrenolone, hydrocortisone sodium succinate, methylprednisolone sodium succinate, prednisolone phosphate sodium, triamcinolone acetonide, hydroxydione sodium, spironolactone, oxandrolone, oxymetholone, prometholone, testosterone cypionate, testosterone phenylacetate, estradiol cypionate, and norethynodrel, analogs thereof, or combinations thereof.

Active agents used with the invention can specifically include proteins, protein fragments, peptides, polypeptides, and the like. By way of example, peptides can include glycosylated proteins, antibodies (both monoclonal and polyclonal), antibody derivatives (including diabodies, f(ab) fragments, humanized antibodies, etc.), cytokines, growth factors, receptor ligands, enzymes, and the like.

### Substrates and Devices

Embodiments of the invention can be used to coat many different types of substrates including many different types of devices, such as medical devices. Medical devices can include both implantable devices (chronically and transiently implantable) and non-implantable medical devices. Embodiments of the invention can also be used to coat implantable medical devices that are designed to be explanted (or removed from the patient) at a point in time after they are first implanted.

Embodiments of the invention can be used with implantable, or transitorily implantable, devices including, but not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), catheters (including arterial, intravenous, blood pressure, stent graft, etc.), valves (e.g., polymeric or carbon mechanical valves, tissue valves, valve designs including percutaneous, sewing cuff, and the like), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuro-aneurysm treatment coils, and neuropatches; ear nose and throat devices such as nasal buttons, nasal and airway splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; biosensor devices including glucose sensors, cardiac sensors, intra-arterial blood gas sensors; oncological implants; and pain management implants.

Classes of suitable non-implantable devices can include dialysis devices and associated tubing, catheters, membranes, and grafts; autotransfusion devices; vascular and surgical devices including atherectomy catheters, angiographic catheters, intraaortic balloon pumps, intracardiac suction devices, blood pumps, blood oxygenator devices (including tubing and membranes), blood filters, blood temperature monitors, hemoperfusion units, plasmapheresis units, transition sheaths, dialators, intrauterine pressure devices, clot extraction catheters, percutaneous transluminal angioplasty catheters, electrophysiology catheters, breathing circuit connectors, stylets (vascular and non-vascular), coronary guide wires, peripheral guide wires; dialators (e.g., urinary, etc.); surgical instruments (e.g. scalpels and the like); endoscopic devices (such as endoscopic surgical tissue extractors, esophageal stethoscopes); and general medical and medically related devices including blood storage bags, umbilical tape, membranes, gloves, surgical drapes, wound dressings, wound management devices, needles, percutaneous closure devices, transducer protectors, pessary, uterine bleeding patches, PAP brushes, clamps (including bulldog clamps), cannulae, cell culture devices, materials for in vitro diagnostics, chromatographic support materials, infection control devices, colostomy bag attachment devices, birth control devices; disposable temperature probes; and pledgets.

In some aspects, embodiments of the invention can be utilized in connection with ophthalmic devices. Suitable ophthalmic devices in accordance with these aspects can provide bioactive agent to any desired area of the eye. In some aspects, the devices can be utilized to deliver bioactive agent to an anterior segment of the eye (in front of the lens), and/or a posterior segment of the eye (behind the lens). Suitable ophthalmic devices can also be utilized to provide bioactive agent to tissues in proximity to the eye, when desired.

In some aspects, embodiments of the invention can be utilized in connection with ophthalmic devices configured for placement at an external or internal site of the eye. Suitable external devices can be configured for topical administration of bioactive agent. Such external devices can reside on an external surface of the eye, such as the cornea (for example, contact lenses) or bulbar conjunctiva. In some embodiments, suitable external devices can reside in proximity to an external surface of the eye.

Devices configured for placement at an internal site of the eye can reside within any desired area of the eye. In some aspects, the ophthalmic devices can be configured for placement at an intraocular site, such as the vitreous. Illustrative intraocular devices include, but are not limited to, those described in U.S. Pat. No. 6,719,750 B2 (“Devices for Intraocular Drug Delivery,” Varner et al.) and U.S. Pat. No. 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and U.S. Application Pub. Nos. 2005/0276837 (filed Aug. 15, 2005, Anderson et al.), U.S. Ser. No. 11/204,271 (filed Aug. 15, 2005, Anderson et al.), U.S. Ser. No. 11/203,981 (filed Aug. 15, 2005, Anderson et al.), U.S. Ser. No. 11/203,879 (filed Aug. 15, 2005, Anderson et al.), U.S. Ser. No. 11/203,931 (filed Aug. 15, 2005, Anderson et al.); and related applications.

In some aspects, the ophthalmic devices can be configured for placement at a subretinal area within the eye. Illustrative ophthalmic devices for subretinal application include, but are not limited to, those described in U.S. Patent Publication No. 2005/0143363 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); U.S. application Ser. No. 11/175,850 (“Methods and Devices for the Treatment of Ocular Conditions,” de Juan et al.); and related applications.

Suitable ophthalmic devices can be configured for placement within any desired tissues of the eye. For example, ophthalmic devices can be configured for placement at a subconjunctival area of the eye, such as devices positioned extrasclerally but under the conjunctiva, such as glaucoma drainage devices and the like.

In some embodiments, elution control coatings as described herein can be formed in the absence of a substrate. By way of example, the elution control coating may take on a form, such as a bead or a film, that is not disposed on a substrate.

It should be noted that, as used in this specification, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

The present invention may be better understood with reference to the following examples. These examples are intended to be representative of specific embodiments of the invention, and are not intended as limiting the scope of the invention.

### EXAMPLES

### Example 1

### Effect of PVP Additive on Elution Rate of Estradiol

A control coating solution was prepared by mixing polyethylene-co-vinyl acetate (PEVA), poly-n-butylmethacrylate (PBMA), and estradiol (β-estradiol, Sigma-Aldrich Corp., St. Louis, Mo.) in a solvent of 100% tetrahydrofuran (THF) in proportions to reach a total solids concentration of 40 mg/ml (30% estradiol/20% PEVA/50% PBMA).

A first test coating solution (“1% PVP K30”) was formed by first mixing polyethylene-co-vinyl acetate (PEVA), poly-n-butylmethacrylate (PBMA), and estradiol together in a solvent of 100% tetrahydrofuran (THF). Next, polyvinylpyrrolidone (K30, m.w. ˜40,000) (BASF, Florham Park, N.J.) was mixed with isopropyl alcohol (IPA) and the resulting solution was mixed in with the solution of PBMA, PEVA, and estradiol. The resulting coating solution had a total solids concentration of 40 mg/ml (30% estradiol/19.5% PEVA/49.5% PBMA/1% PVP) in a solvent of 95% THF and 5% IPA.

A second test coating solution (“10% PVP K30”) was formed by first mixing polyethylene-co-vinyl acetate (PEVA), poly-n-butylmethacrylate (PBMA), and estradiol together in a solvent of 100% tetrahydrofuran (THF). Next, polyvinylpyrrolidone (K30, m.w. ˜40,000) (BASF, Florham Park, N.J.) was mixed with isopropyl alcohol (IPA) and the resulting solution was mixed in with the solution of PBMA, PEVA, and estradiol. The resulting coating solution had a total solids concentration of 40 mg/ml (30% estradiol/15% PEVA/45% PBMA/10% PVP) in a solvent of 90% THF and 10% IPA.

A third test coating solution (“1% PVP K90”) was formed by first mixing polyethylene-co-vinyl acetate (PEVA), poly-n-butylmethacrylate (PBMA), and estradiol together in a solvent of 100% tetrahydrofuran (THF). Next, polyvinylpyrrolidone (K90, m.w. ˜360,000) (BASF, Florham Park, N.J.) was mixed with isopropyl alcohol (IPA) and the resulting solution was mixed in with the solution of PBMA, PEVA, and estradiol. The resulting coating solution had a total solids concentration of 40 mg/ml (30% estradiol/19.5% PEVA/49.5% PBMA/1% PVP) in a solvent of 90% THF and 10% IPA.

A fourth test coating solution (“10% PVP K90”) was formed by first mixing polyethylene-co-vinyl acetate (PEVA), poly-n-butylmethacrylate (PBMA), and estradiol together in a solvent of 100% tetrahydrofuran (THF). Then, polyvinylpyrrolidone (PVP) (K90, m.w. ˜360,000) (BASF, Florham Park, N.J.) was mixed with isopropyl alcohol (IPA) and the resulting solution was mixed in with the solution of PBMA, PEVA, and estradiol. The resulting coating solution had a total solids concentration of 40 mg/ml (30% estradiol/15% PEVA/45% PBMA/10% PVP) in a solvent of 90% THF and 10% IPA.

The control coating solution and the test coating solutions were then applied to stents using an ultrasonic spray coating system. Ultrasonic spray techniques are disclosed in U.S. Published Application 2004/0062875 (Chappa et al.), the contents of which are herein incorporated by reference. Specifically, each solution was applied onto an ultrasonic spray nozzle at a rate of approximately 0.2 mls/minute. The ultrasonic spray nozzle was operating at 0.5 watts. The humidity was kept at approximately 30% relative humidity at ambient temperature. After coating, the stents were then allowed to dry over night under ambient conditions. The details for each group of stents coated are shown below in Table 1.

TABLE 1

| | Total Coating Wt. | |
|---|---|---|
| Group | (μg) | Drug Load (μg) |
| Control (N = 3) | 1041.00 | 312.33 |
| 1% PVP K30 (N = 3) | 1011.33 | 303.33 |
| 10% PVP K30 (N = 2) | 1041.50 | 312.50 |
| 1% PVP K90 (N = 3) | 1055.67 | 316.67 |
| 10% PVP K90 (N = 3) | 1167.33 | 350.67 |

The elution rate of estradiol from the stents was then assessed. Specifically, the stents were individually placed in a phosphate buffered saline (PBS) solution including TWEEN20 at a concentration of 0.01 M. At predetermined intervals, samples of the PBS solution were then analyzed using HPLC to determine the amount of estradiol. The averaged cumulative elution data for each group of stents are shown in Table 2 and FIG.

TABLE 2

| | | 1% PVP K30 | 10% PVP K30 | 1% PVP K90 | 10% PVP K90 |
|---|---|---|---|---|---|
| Hours | Control (μg) | (μg) | (μg) | (μg) | (μg) |
| 2 | 33.3038 | 44.4565 | 130.4910 | 61.1791 | 126.6476 |
| 25 | 125.6201 | 168.9980 | 282.1808 | 234.5956 | 264.8385 |
| 48 | 182.2591 | 229.1741 | 290.2960 | 285.2461 | 278.8999 |
| 72 | 228.0648 | 260.6347 | 290.2960 | 301.9814 | 282.1711 |
| 160 | 282.1170 | 284.2213 | 290.2960 | 308.6558 | 282.9404 |

This example shows that additives such as PVP can be used to modify the elution kinetics of an active agent from an elution control coating. In specific, this example shows that although the addition of PVP did not substantially affect the total amount of the active agent released, PVP did function to increase the elution rate of the active agent.

### Example 2

### Effect of PVP Additive on Elution Rate of Active Agent with Limited Solubility in Both Polar and Non-Polar Solvents

An active agent solution is formed by mixing an active agent in a solvent mixture of 80/20 (vol %) acetonitrile/water in proportions to reach a total solids concentration of 20 mg/ml. The active agent is one with a solubility of less than about 2 mg/ml in 100% water and a solubility of less than about 2 mg/ml in 100% chloroform.

A control polymer solution is prepared by mixing polyethylene-co-vinyl acetate (PEVA), poly-n-butylmethacrylate (PBMA) in equal proportions in a solvent of chloroform to reach a total solids concentration of 40 mg/ml.

A test polymer solution (test) is prepared by mixing polyethylene-co-vinyl acetate (PEVA), poly-n-butylmethacrylate (PBMA), and polyvinylpyrrolidone (K30, m.w. ˜40,000) (BASF, Florham Park, N.J.) in a solvent of chloroform to reach a total solids concentration of 40 mg/ml (˜47 wt. % PEVA, 47 wt. % PBMA, 6 wt. % PVP).

The coating solutions are then applied to stents using a dual-spray ultrasonic spray coating system. Specifically, for a first set of stents (control), the active agent solution is applied from a first spray head while the control polymer solution is applied simultaneously from a second spray head. For a second set of stents (test), the active agent solution is applied from a first spray head while the test polymer solution is applied simultaneously from a second spray head. Dual spray ultrasonic coating systems and methods are disclosed in U.S. Published Application 2007/0128343 (Chappa et al.), the contents of which are herein incorporated by reference. After coating, the stents are allowed to dry over night under ambient conditions. The first set of stents (control) are determined to have a coating containing approximately 30 wt. % active agent, 35 wt. % PEVA, and 35 wt. % PBMA. The second set of stents (test) are determined to have a coating containing approximately 30 wt. % active agent, 33 wt. % PEVA, 33 wt. % PBMA, and 4 wt. % PVP.

The elution rate of the active agent from the stents is then assessed. Specifically, the stents are individually placed in a phosphate buffered saline (PBS) solution including TWEEN20 at a concentration of 0.01 M. At predetermined intervals, samples of the PBS solution are then analyzed using HPLC to determine the amount of the active agent. The data will show that the active agent eluted faster from the second set of stents (test) in comparison with the first set of stents (control).

This example will show that additives such as PVP can be used to increase the elution rates of active agents having limited solubility in both polar and non-polar solvents. This example will specifically show that additives such as PVP can be used to increase the elution rates of active agents having a solubility in water of less than about 2 mg/ml and a solubility in chloroform of less than about 2 mg/ml.

1. A method of forming an elution control coating with accelerated release kinetics comprising:
disposing a hydrophobic polymer, an additive comprising polyvinylpyrrolidone, and an active agent having limited solubility in water, on a substrate to form the elution control coating, the elution control coating having a solids concentration of at least about 0.1 wt. % of polyvinylpyrrolidone.

2. The method of claim 1, the active agent comprising estradiol.

3. The method of claim 1, the active agent having a solubility in water of less than about 2 mg/ml and a solubility in chloroform of less than about 2 mg/ml.

4. The method of claim 1, the coating solution having a solids concentration of at least about 5.0 wt. % of polyvinylpyrrolidone.

5. The method of claim 1, the hydrophobic polymer comprising a polymer with a solubility parameter of less than about 11.0 (cal/cm³)^{1/2}.

6. The method of claim 1, the hydrophobic polymer comprising poly(ethylene-co-vinyl acetate) and poly(n-butyl methacrylate).

7. An elution control coating comprising:
a polymeric matrix;
at least about 0.1 wt. % of an additive dispersed within the polymeric matrix, the additive comprising polyvinylpyrrolidone or a copolymer thereof; and
an active agent dispersed within the polymeric matrix, the active agent having limited solubility in water.

8. The elution control coating of claim 7, the additive comprising polyvinylpyrrolidone homopolymer.

9. The elution control coating of claim 7, the active agent comprising a steroid.

10. The elution control coating of claim 7, the active agent comprising estradiol.

11. The elution control coating of claim 7, the active agent having a solubility in water of less than about 2 mg/ml and a solubility in chloroform of less than about 2 mg/ml.

12. The elution control coating of claim 7, the active agent insoluble in water.

13. The elution control coating of claim 7, the polymeric matrix comprising a hydrophobic polymer.

14. The elution control coating of claim 7, the polymeric matrix comprising a polymer with a solubility parameter of less than about 11.0 (cal/cm³)^{1/2}.

15. The elution control coating of claim 7, the polymeric matrix comprising poly(ethylene-co-vinyl acetate) and poly(n-butyl methacrylate).

16. The elution control coating of claim 7, the polymeric matrix comprising an uncross-linked polymeric matrix.

17. The elution control coating of claim 7, the polymeric matrix configured to resist fracturing under in vivo conditions for at least about six weeks.

18. The elution control coating of claim 7, the coating configured to elute the active agent at a rate faster than an otherwise identical elution control coating lacking the additive.

19. The elution control coating of claim 7, comprising a solids concentration of at least about 5.0 wt. % of polyvinylpyrrolidone.

20. A method of increasing the elution rate of an active agent from a coating comprising:
combining a matrix forming polymer, an active agent having limited solubility in water, and a solvent together to form a coating solution;
adding polyvinylpyrrolidone to the coating solution in an amount sufficient for the coating solution to have a solids concentration of at least about 0.1 wt. % of polyvinylpyrrolidone, and
disposing the coating solution on a substrate.

21. A method of forming an elution control coating comprising:
depositing a polymeric matrix onto a substrate;
depositing an active agent onto the substrate, the active agent having limited solubility in water; and
depositing polyvinylpyrrolidone onto the substrate in an amount sufficient for the elution control coating to comprise at least about 0.1 wt. % (solids) of polyvinylpyrrolidone.
